SEQUENCE LISTING (3 sequences)

SEQ ID NO: 1 - protein, 122 aa, Escherichia coli
MSFKKIIKAFVIMAALVSVQAHAGASQFFKDNCNRTTASLVEGVELTKYISDINNNTDGMYVVSSTGGVWRISRAKDYPDNVMTAEMRKIAMAAVLSGMRVNMCASPASSPNVIWAIELEAE

SEQ ID NO: 2 - protein, 99 aa, Escherichia coli
GASQFFKDNCNRTTASLVEGVELTKYISDINNNTDGMYVVSSTGGVWRISRAKDYPDNVMTAEMRKIAMAAVLSGMRVNMCASPASSPNVIWAIELEAE

SEQ ID NO: 3 - protein, 99 aa, Escherichia coli
GASQFFKDNCNRITASLVEGVELTKYISDINNNTDGMYVVSSTGGVWRISRAKDYPDNVMTAEMRKIAMAAVLSGMRVNMCASPASSPNVIWAIELEAE

(19) United States
(12) Patent Application Publication
(10) Pub. No.: US 2015/0366963 A1
(43) Pub. Date: Dec. 24, 2015

(54) METHODS FOR ENHANCING AN IMMUNE RESPONSE TO AN ANTIGEN

(71) Applicants: THE RESEARCH FOUNDATION FOR THE STATE UNIVERSITY OF NEW YORK, Amherst, NY (US); HEALTH RESEARCH, INC., Menands, NY (US)

(72) Inventors: Terry D. CONNELL, Clarence Center, NY (US); Nicholas MANTIS, Williamstown, MA (US); Christopher GREENE, Tonawanda, NY (US)

(21) Appl. No.: 14/766,047

(22) PCT Filed: Feb. 6, 2014
(86) PCT No.: PCT/US14/15114

§ 371 Date: Aug. 5, 2015

Related U.S. Application Data
(60) Provisional application No. 61/761,260, filed on Feb. 6, 2013.

(51) Int. Cl. A61K 39/39

(52) U.S. Cl. CPC A61K 39/39; A61K 2039/55544

(57) ABSTRACT

Provided are methods and kits for enhancing an immune response to an antigen in an individual comprising administering to the individual an effective amount of (a) LT-IIb or (b) LT-IIb(T13I) enterotoxin, and (c) an antigen, whereby the LT-IIb or LT-IIb(T13I) enterotoxin acts as an adjuvant to enhance the generation of antigen specific neutralizing antibodies.

### CROSS-REFERENCE TO RELATED APPLICATIONS

This application claims priority to U.S. Provisional Application No. 61/761,260, filed on Feb. 6, 2013, the disclosure of which is incorporated herein by reference.

### STATEMENT REGARDING FEDERALLY SPONSORED RESEARCH

This invention was made with government support under grant number DE013833 from the National Institutes of Health. The government has certain rights in the invention.

### FIELD OF THE INVENTION

This invention relates generally to the field of vaccine adjuvants.

### BACKGROUND OF THE INVENTION

There is a growing need to develop safe and effective vaccines against a range of biological threat agents. One of the major concerns is ricin, a potent, bipartite toxin derived from the castor bean plant [Ricinus communis]. Ricin toxin, is easily isolated from castor beans using simple enrichment steps and is extremely toxic to humans whether injected, inhaled, or ingested. Due to its potent toxicity and ease of dissemination, the Centers for Disease Control and Prevention (CDC) has designated ricin to be a Category B toxin. It is estimated that a single molecule of ricin is sufficient to kill a mammalian cell.

Ricin is a member of the type II ribosome inactivating protein family of toxins. Ricin is a 65 kilodalton (kDa) polypeptide toxin comprised of two dissimilar polypeptide chains (an A-chain and a B-chain) held together by a disulfide bond. The ricin toxin A subunit (RTA) is an RNA N-glycosidase that depurinates a conserved adenosine residue located within the sacrin/ricin loop of the eukaryotic 28s ribosomal RNA (rRNA). Depurination of this residue results in an immediate cessation of ribosome progression, which subsequently inhibits protein synthesis. The ricin toxin B subunit (RTB) binds with micromolar affinity to α(1-3)-linked galactose and N-acetylgalactosamine residues that are expressed on the surface of all mammalian cell types. Binding of RTB to these receptors mediates internalization and retrograde transport of the ricin holotoxin to the endoplasmic reticulum (ER). In the ER, RTA dissociates from RTB and is then retrotranslocated across the ER membrane into the cytosol where it gains access to rRNA targets. In addition to ribosome inactivating properties, ricin also elicits vascular leak syndrome (VLS), which primarily afflicts endothelial cells.

Although a considerable amount of effort has been expended on developing post-exposure treatments for ricin intoxication, immunization against ricin remains the most reasonable and reliable method to ensure long-lasting protection for military forces, first responders, and research personnel. Past and current efforts to develop a safe, effective vaccine include ricin toxoid, deglycoslated RTA, and a truncated form of RTA known as RVEc. One of the most promising candidates, however, is RiVax™. RiVax™ is a full-length recombinant derivative of RTA in which a tyrosine to alanine substitution at amino acid position 80 and a valine to methionine substitution at amino acid position 76 were engineered to eliminate the RNA N-glycosidase and VLS activities of the protein. Previous studies in mice and rabbits demonstrated that RiVax™ is safe and immunogenic when administered by the intramuscular (i.m.) and intradermal (i.d.) routes. In a Phase I clinical trial, however, the vaccine was not totally effective. Not all participants responded to RiVax™ when employed as an i.m. vaccine in that trial. In an attempt to enhance the vaccine's efficacy, alum was employed as a potential adjuvant in a subsequent trial. Data from this second Phase I trial, in which individuals received three i.m. immunizations over a span of 26 weeks, demonstrated that alum had the capacity to enhance immune responses against RiVax™. Yet, the levels of anti-RiVax™ Ab in this trial were neither robust nor long lasting, especially in the group that received the lowest dose of vaccine. Rather, a number of subsequent booster injections of RiVax™ in combination with alum was required before detectable levels of anti-RiVax™ Ab were detected in the sera of the two low dose′ groups. These data underscored the need to identify new adjuvants to boost the efficacy of RiVax™.

Some of the most potent mucosal adjuvants described to date belong to the families of bacterial heat-labile enterotoxins (HLT). The HLT expressed by enterotoxigenic Escherichia coli (LT, LT-IIa, LT-IIb, LT-IIc) and by Vibrio cholerae [cholera toxin, (CT)] are composed of a single, enzymatically active A subunit that is non-covalently bound to a pentameric array of B subunits. The A subunit of HLT is a potent ADP-ribosylase that targets the G_{sα} regulatory protein of the adenylate cyclase system. The B pentamer of HLT mediates binding of the holotoxin to ganglioside receptors, which are a family of cell surface glycolipids found ubiquitously on mammalian cells. Each HLT binds to a unique ganglioside or with varying affinity to members of a subset of gangliosides. For example, CT and LT bind avidly to ganglioside GM1. In contrast, LT-IIb binds with highest affinity to GD1a and less to GM2 and GM3.

A number of studies revealed that LT-IIb is a potent mucosal and systemic adjuvant when administered at mucosal surfaces. Intranasal (i.n.) immunization of mice with model antigens (Ag) in combination with LT-IIb induces robust anti-Ag immune responses at local mucosal sites, distal mucosal sites, and systemically. The enthusiasm for the potential use of LT-IIb and other HLT as intranasal adjuvants has been reduced, however, by concerns of the inherent toxicity of the molecules. For that reason, considerable effort has been expended toward developing safe and effective HLT mutants that lack toxicity, yet retain the potent adjuvant properties of the native proteins. Substitution of the threonine at amino acid position 13 in the B polypeptide of LT-IIb with an isoleucine [LT-IIb(T13I)] dramatically reduced the binding affinity of that mutant HLT to its respective ganglioside receptors, which in turn dramatically reduced the HLT's toxicity to levels that were undetectable by standard bioassays. The reduced binding affinity of the mutant HLT for gangliosides did not ablate the capacity of LT-IIb(T13I) to bind to various immune cells including macrophages, CD8+ T cells, CD4+ T cells, and B cells that were bound by wt LT-IIb. It is not surprising, therefore, that when used as an i.n. adjuvant, LT-IIb(T13I) exhibited immunomodulatory properties that were similar to those observed for native LT-IIb. Unfortunately, concerns have been raised for the use of any HLT as i.n. adjuvants.

### BRIEF SUMMARY

The present disclosure provides compositions and methods for enhancing an immune response to an antigen in an individual comprising administering to the individual an effective amount of (a) LT-IIb or (b) LT-IIb(T13I) enterotoxin, and (c) an antigen, whereby the LT-IIb or LT-IIb(T13I) enterotoxin acts as an adjuvant to enhance the immune response to the antigen.

In the present disclosure, we unexpectedly observed that by administering the adjuvant and the antigen via the intradermal route results in a preferential increase in antigen neutralizing antibodies. When the antigen is a toxin, these antibodies are referred to as toxin neutralizing antibodies. Thus, the terms “antigen neutralizing antibodies”, “antigen specific neutralizing antibodies”, toxin neutralizing antibodies” and “toxin specific neutralizing antibodies” are used interchangeably. Specific toxin neutralizing antibodies (such as ricin neutralizing antibodies) comprise only a small percent of the total antibodies generated against ricin but are considered critical for protection against ricin poisoning. In mice, this number is considered to be about 1%. Neutralizing antibodies or toxin neutralizing antibodies are defined as a subset of the total anti-toxin antibodies that upon binding to their epitope on the toxin, eliminate the ability of the toxin to exert it's toxic effects on cells (such as mammalian cells). Therefore the ability to produce these specific “ricin neutralizing antibodies” is desired. The present disclosure provides a method to enhance the proportion of this small subset of critical toxin neutralizing antibodies after intradermal immunization. Thus, in the immune response generated after administration of the adjuvant and the antigen, not only did the titer of antibodies increase, the ratio of neutralizing antibodies to total antibodies also increased. Such an increase was not observed when the adjuvant and the antigen were administered via a mucosal route.

In one embodiment, the present disclosure provides a method for enhancing an immune response to an antigen (such as a toxin) in an individual comprising intradermally administering to the individual an effective amount of an enterotoxin adjuvant selected from the group consisting of LT-IIb or LT-IIb (T13I) and an antigen (such as a toxin) whereby the immune response to the antigen in enhanced.

In one embodiment, the present disclosure provides a method for enhancing an immune response to a toxin in an individual comprising intradermally administering to the individual an effective amount of an adjuvant selected from the group consisting of LT-IIb and LT-IIb(T13I), and a toxin selected from the group consisting of ricin and anthrax or a non-toxic subunit thereof, or a derivative thereof, wherein the intradermal administration results in generation of toxin-neutralizing antibodies.

In one embodiment, adjuvant is selected from the group consisting of LT, LT-IIa, LT-IIb, LT-IIc or their mutants. In one embodiment, the antigen is a toxin. In one embodiment, the toxin is ricin or anthrax, or a subunit thereof, or a non-toxic variant of the subunit or the toxin.

In one aspect, the present disclosure provides a pharmaceutical kit for intradermal delivery of an adjuvant selected from the group consisting of LTII-a, LT-IIb, LT-IIb(T13I), and LT-IIc, an antigen, and an intradermal delivery device. The adjuvant and the antigen may be provided as a single composition which may be prefilled in the intradermal delivery device.

### BRIEF DESCRIPTION OF THE FIGURES

FIG. 1. LT-IIb and LT-IIb(T13I) enhance the immune response to RiVax™ when co-administered by the i.d. route. Mice were immunized intradermally with 5.0 μg of RiVax™ alone or with 1.0 μg of LT-IIb or LT-IIb(T13I) on days 0, 10, and 20. (A) Level of Ag-specific IgG Abs in sera from immunized mice on days 7, 17, and 27. (B) Level of RiVax™-specific IgG and IgA Abs obtained on day 27 in BAL, salivary, and fecal samples from immunized mice. Data presented as the arithmetic mean with error bars denoting one S.E.M. (n=5). Key: *, p<0.05; **, p<0.01; ***, p<0.001 compared to RiVax™ alone.

FIG. 2. I d Immunization of RiVax™ with LT-IIb or LT-IIb(T13I) enhances ricin-neutralizing Ab production. Sera from immunized mice were assessed for the capacity to neutralize ricin in a Vero cell cytotoxicity assay. Ricin (10 ng/mL) was incubated with serum for 30 min and the mixture was applied in triplicate to Vero cells grown in 96-well microtiter plates for 2 h at 37° C. After washing, fresh media was applied and cell viability was assessed 48 h later. Data shown is representative for each group of immunized animals (n=5) with the error bars denoting one S.E.M. of the average of three replicate wells.

FIG. 3. Administration of LT-IIb(T13I) by the i.d. route enhances protective immunity to ricin. Mice were immunized intradermally on days 0, 10, and 20 with 0.5 μg of RiVax™ in the presence or absence of 1.0 μg of LT-IIb(T13I) and challenged 14 days after the final immunization with 10 LD50 of ricin. (A) Survival of immunized mice after i.p. challenge with ricin. (B) Blood glucose levels of immunized mice during challenge with ricin. Data presented as the arithmetic mean with error bars denoting one S.E.M. (n=5). Key: *, p<0.05; **, p<0.01 compared to RiVax™; †, p<0.05 compared to Day 0.

FIG. 4. LT-IIb(T13I) is superior to alum at enhancing immune responses to RiVax™ when administered intradermally. Mice were immunized intradermally on days 0, 10, and 20 with 0.5 μg of RiVax™, RiVax™ adsorbed to aluminum adjuvant, or RiVax™ combined with 1.0 μg of either LT-IIb or LT-IIb(T13I). (A) Level of RiVax™-specific serum IgG Ab from days 17, 27, 60, 90, and 120. (B) RiVax™-specific IgG subclass analysis from the sera of immunized mice taken on day 90. Data are presented as the arithmetic mean with error bars denoting one S.E.M. (n=5). Key: *, p<0.05 compared to RiVax™ (panel A) or IgG2a and IgG2b (panel B); **, p<0.01; ***, p<0.001 compared to RiVax™; #, p<0.05 compared to RiVax™+alum.

FIG. 5. LT-IIb(T13I) is minimally inflammatory in comparison to LT-IIb. Mice were immunized by the i.d. route with 5.0 μg of RiVax™, RiVax™ adsorbed to aluminum adjuvant, or RiVax™ combined with 1.0 μg of either LT-IIb or LT-IIb(T13I). (A) Gross morphologic photos and H&E stained micrographs of typical inflammation at the site of injection on day 7 post-immunization (n=5). (B) Level of inflammatory induration at the injection site. Data are presented as the arithmetic mean with error bars denoting one S.E.M. (n=5). *, p<0.05; **, p<0.01; ***, p<0.001 compared to LT-IIb(T13I).

FIG. 6. LT-IIb(T13I) enhances immune cell accumulation at the injection site. Paraffin-embedded sections of skin from immunized mice underwent Ag retrieval and were stained using a rat anti-mouse CD45 primary Ab followed by chicken anti-rat Alexa647 (Cy-5) Ab. Collagen 1 was stained for using a rabbit anti-rat collagen 1 Ab followed by chicken anti-rat Alexa488 (FITC) Ab. DAPI was used as a nuclear stain. (A) Representative low-magnification image of a stained skin section from an immunized mouse. (B) Representative fluorescent micrographs of stained sections. The lower panel is a higher magnification of the section outlined by the white box in the upper photograph. (C) Level of CD45+ immune cells normalized to the total number of nuclei counted using Axiovision and ImageJ software from 15 random fluorescent micrographs per section. Data are presented as the arithmetic mean with error bars denoting one S.E.M (n=3). Key: *, p<0.05 compared to RiVax™ alone group; # p<0.05 compared to LT-IIb.

FIG. 7. Immunization with LT-IIb(T13I) by the i.n. route enhances RiVax™-specific Ab production. Mice were immunized intranasally with 5.0 μg of RiVax™ alone or with 1.0 μg of LT-IIb or LT-IIb(T13I) on days 0, 10, and 20. (A) Level of RiVax™-specific IgG Ab in sera from immunized mice on days 7, 17, and 27. (B) Level of RiVax™-specific IgG and IgA Ab obtained on day 27 from lung lavage, saliva, and fecal samples from immunized mice. Data are presented as the arithmetic mean with error bars denoting one S.E.M. (n=5). *, p<0.05; **, p<0.01; ***, p<0.001 compared to RiVax™.

FIG. 8. I.n. immunization of RiVax™ with LT-IIb(T13I) enhances protective immunity to ricin. Mice were immunized intranasally on days 0, 10, 20, and 34 with 0.5 μg of RiVax™ in the presence or absence of 1.0 μg of LT-IIb or LT-IIb(T13I) and challenged 14 days after the final immunization with 10 LD50 of ricin. (A) Survival of mice after i.p. challenge with ricin. (B) Blood glucose levels of immunized mice during ricin challenge. Data are presented as the arithmetic mean with error bars denoting one S.E.M. (n=5).

### DETAILED DESCRIPTION OF THE INVENTION

The following description will provide specific examples of the present invention. Those skilled in the art will recognize that routine modifications to these embodiments can be made which are intended to be within the scope of the invention.

All references to LT-IIb are meant to describe LT-IIb enterotoxins (oligomeric proteins) comprising a wild-type A polypeptide of LT-IIb that is noncovalently coupled to a pentameric array of wild-type B polypeptides, wherein the B polypeptide sequence is designated by SEQ ID NO:2. The B polypeptide sequence with the leader sequence included is designated as SEQ ID NO:1. All references to LT-IIb(T13I) are meant to describe LT-IIb enterotoxins (oligomeric proteins) comprising a wild-type A polypeptide of LT-IIb that is noncovalently coupled to a pentameric array of mutant B polypeptides, wherein the mutant B polypeptide sequence is designated by SEQ ID NO:3. The complete sequence of LT-IIb is available in Connell et al., 1995, Molecular Microbiology, 16:21-31. The sequences of LT-IIb subunits including Subunit A are provided in Pickett et al., 1989, J. Bacteriol. 171(9):4945, which sequences is incorporated herein by reference.

We present here methods for enhancing an immune response to an antigen in an individual comprising administering to the individual an effective amount of (a) LT-IIb or (b) LT-IIb(T13I) enterotoxin, and (c) an antigen, whereby the LT-IIb or LT-IIb(T13I) enterotoxin acts as an adjuvant to enhance the immune response to the antigen.

In some embodiments, administration takes place via a parenteral route: for example, the intradermal route. In other embodiments, administration takes place via a mucosal route: for example, the intranasal route.

In one embodiment, the antigen is a toxin. In some embodiments, the antigen is ricin toxin or a subunit or derivative thereof such as a non-toxic mutant, which is effective in generating an immune response against ricin. For example, the ricin antigen may be selected from the group consisting of ricin toxoid, ricin toxin A subunit or recombinant derivatives thereof (e.g. RiVax™), deglycosylated RTA, and a truncated form of RTA known as RVEc. In one embodiment, it is a full-length recombinant derivative of RTA in which there is a tyrosine to alanine substitution at amino acid position 80 and a valine to methionine substitution at amino acid position 76. (Smallshaw et al., 2002, Vaccine, 20:3422-3427; Smallshaw et al., 2005, Vaccine, 23:7459-7469; Vitetta et al., 2006, PNAS, 103:2268-2273). This eliminates the RNA N-glycosidase and VLS activities of the protein. This is commercially available under the trade name RiVax™ (Soligenix, Princeton, N.J.).

In one embodiment, the toxin is anthrax or a subunit or derivative thereof which is effective in generating an immune response against anthrax. For example, anthrax protective antigen (PA) portion of the anthrax toxin could be used for immunizations. In one embodiment, the composition may comprise a plurality of antigens and/or a plurality of adjuvants.

The adjuvant and the toxin (or other antigens) may be administered in a single shot or may be followed up by booster shots. Such administration regimens are routine and well within the purview of those skilled in the art.

In some embodiments, the immune response is measured by the proportion of antigen-specific antibodies that possess neutralizing activity. To that end, some embodiments of the present invention comprise methods to enhance the production of neutralizing antibodies to an antigen in an individual comprising administering to the individual an effective amount of (a) LT-IIb or (b) LT-IIb(T13I) enterotoxin, and (c) an antigen, whereby the LT-IIb or LT-IIb(T13I) enterotoxin acts as an adjuvant to enhance the production of antigen-specific neutralizing antibodies. In some examples, administration via a parenteral route such as the intradermal route may be employed. In other examples, mucosal (e.g. intranasal) administration may be used.

In some embodiments, the antigen may be administered simultaneously with LT-IIb or LT-IIb(T13I). In other embodiments, the antigen and either (a) LT-IIb or (b) LT-IIb(T13I) may be administered sequentially. In other embodiments, LT, LT-IIa or LT-IIc may be used.

In some embodiments, the antigen and either (a) LT-IIb or (b) LT-IIb(T13I) may be administered as a fused molecule. In other embodiments, they may be administered as a chemically conjugated molecule. In still other embodiments, the antigen and either (a) LT-IIb or (b) LT-IIb(T13I) may be administered as a chimeric molecule.

In one embodiment, the immune response is comprised of a high proportion of antigen-specific neutralizing antibodies. In one embodiment, the high proportion of neutralizing antibodies is determined based on a comparison to those elicited by administering (a) the antigen alone or (b) the antigen and alum. In one embodiment, the high proportion of neutralizing antibodies is determined based on a comparison to those elicited by administration of the adjuvant and the antigen via a mucosal (such as intranasal) route. In one embodiment, the antigen-specific neutralizing antibodies confer a protective effect to the individual upon subsequent challenge with an antigen-related toxin.

It was observed that LT-IIb(T13I) also unexpectedly and preferentially stimulated the production of antigen-neutralizing Ab, which was correlated with a strong augmentation in protective immunity to antigen challenge.

In one embodiment, the immunogenic composition comprises effective amounts of LT-IIb(T13I) and a ricin antigen. In another embodiment, the immunogenic composition comprises effective amounts of LT-IIb(T13I) and an anthrax antigen. The immunogenic compositions are in such amounts that they will elicit an immune response when administered intradermally, wherein the total antibodies generated have a higher proportion of antigen specific antibodies compared to administration of the composition via the nasal route.

In one embodiment, the method of co-administering (a) LT-IIb or (b) LT-IIb(T13I), and (c) a ricin antigen via the intradermal route elicits an enhanced antigen-specific immune response, wherein the LT-IIb or LT-IIb(T13I) enterotoxin acts as an adjuvant that elicits a high level of antigen-specific neutralizing antibodies that confer a protective effect upon subsequent challenge with ricin.

The compositions may additionally contain one or more pharmaceutically acceptable excipients or vehicles such as water, saline, glycerol, ethanol, etc. The compositions may also contain wetting or emulsifying agents, biological buffers, and the like. A biological buffer is any solution which is pharmacologically acceptable and which provides the adjuvant formulation with the desired pH, i.e., a pH in the physiological range. Examples of buffer solutions include saline, phosphate buffered saline, Tris buffered saline (TBS), Hank's buffered saline (HBS), growth media such as Eagle's Minimum Essential Medium (“MEM”), and the like.

The immunogenic formulations may be delivered intradermally. Suitable devices for intradermal delivery include short needles, microneedles. Other devices including syringe jet injectors which are needle-free may also be used. Intradermal delivery devices such as microneedle patches may also be used. Further, prefilled syringes can facilitate intradermal delivery of small and precise volumes. Examples of suitable intradermal delivery devices can be found in U.S. Pat. Nos. 5,328,483, 5,527,288, 5,599,302, 5,649,912, 5,704,911, 5,893,397, 6,971,999, and 7,410,476. The intradermal delivery devices may be disposable, single use devices such that the entire volume of the device can be delivered.

In one aspect, this disclosure provides a kit comprising an administration device suitable for intradermal administration and an immunogenic formulation comprising an adjuvant and an antigen as described herein. The device may be supplied pre-filled with the immunogenic formulation. In one embodiment, the immunogenic formulation is in a liquid volume smaller than for conventional intramuscular vaccines. For example, the intradermal administration devices may contain a volume of between about 0.05 ml and 0.2 ml. The kit may also contain a needle delivery device suitable for administering the formulation to the dermis.

In one embodiment, this disclosure provides a kit comprising: a) an immunogenic composition comprising an effective amount of LT-IIb(T31I) and a non-toxic variant of a toxin in a pharmaceutically suitable medium; b) an intradermal delivery device; and instructions for use of the delivery device such that the immunogenic composition can be delivered intradermally to an individual. In one embodiment, the intradermal delivery device is prefilled with the immunogenic composition. Thus, the intradermal delivery device may be prefilled with 50-200 μl microliters of a composition comprising LT-IIb(T13I) and a non-toxic variant of ricin such as RiVax and/or a non-toxic variant of anthrax such as the PA.

In one embodiment, the injection volume is from 50 to 200 μl and all integers and ranges therebetween. In another embodiment the injection volume is from 80 to 120 μl. In one embodiment, a single injection is used. In another embodiment, a total of up to 5 injections may be used, including booster injections, which may be given over a period of time. In one embodiment, the amount of toxin antigen is from 1-20 μg per dose and all integers therebetween, and the amount of adjuvant per dose is also from 1 to 20 μg and all integers therebetween. In one embodiment, both the toxin antigen and the adjuvant are about 10 μg per dose (from 9 to 11 μg).

In one aspect, the present disclosure provides a method for administration of an immunogenic formulation described herein. The method comprises the steps of providing an interdermal delivery device comprising the immunogenic formulation and delivering intradermally to an individual, the immunogenic formulation via the delivery device.

### Example 1

This example described enhanced generation of neutralizing antibodies by intradermal administration of a formulation described herein.

### Materials and Methods

Chemicals and Reagents.

Recombinant His-tagged LT-IIb and LT-IIb(T13I), were purified using previously described methods (Nawar et al., Infection and Immunity, March 2005, 73(3):1330-1342, the description of the method is incorporated herein by reference). All preparations were determined to be essentially free of lipopolysaccaride (<0.03 ng/μg protein) using a Limulus amoebocyte assay kit (Charles River Endosafe, Charleston, S.C.). Ricin (RCA-II; Catalog L-1090) was purchased from Vector Laboratories (Burlingame, Calif.). RiVax™TM was provided by Dr. Robert Brey (Soligenix Inc., Princeton, N.J.). Rat anti-mouse CD45 primary Ab (550539) was purchased from BD Pharmingen (San Diego, Calif.). Rabbit anti-rat collagen type I Ab (AB755) was purchased from Chemicon International Inc. (Temecula, Calif.). Chicken anti-rat Alexa647 (A-21472) and chicken anti-rabbit Alexa488 (A-21441) Ab, DAPI (D21490), and SlowFade Gold antifade reagent (S36936) were purchased from Invitrogen (Grand Island, N.Y.). Imject® Alum adjuvant (77161) was purchased from Pierce (Rockford, Ill.). Protease inhibitor cocktail (P50700-1.0) was purchased from Research Products International (Prospect, Ill.).

Mice and Immunizations.

All mice used in this study were housed under conventional, specific pathogen-free conditions and were treated in strict compliance with guidelines established by the Institutional Animal Care and Use Committees (IACUC) at the University at Buffalo and at the Wadsworth Center, New York State Department of Health. Eight to twelve week old, female BALB/c mice were purchased from Harlan Laboratories (Madison, Wis.) or Taconic Laboratories (Hudson, N.Y.). For i d immunizations, groups of 5 mice were first anesthetized with 75 mg/kg of ketamine and 10 mg/kg of xylazine or isoflurane and hair was removed on their dorsum and cleaned with alcohol swabs. Using insulin syringes, mice were intradermally immunized with PBS (vehicle control), RiVax™ (0.5 or 5 μg), or various combinations of RiVax™ with 1.0 μg of LT-IIb, 1.0 μg of LT-IIb(T13I), or adsorbed to Imject® Alum in 10 or 50 μL volumes for low and high doses of RiVax™, respectively. For i.n. immunizations, a well-established mouse mucosal immunization model was employed. Groups of 5 unanesthetized mice were immunized by the i.n. route with PBS (vehicle control), RiVax™ (0.5 or 5.0 μg), or RiVax™ in combination with 1.0 μg of LT-IIb or LT-IIb(T13I) Immunizations were administered in standardized volumes that were applied to both external nostrils (5 μl/naris) with mice being rested for 5 minutes between each nasal administration. The i.n. and i.d. immunization regimens consisted of a primary immunization followed by booster immunizations administered at day 10 and day 20.

Collection of Serum and Secretion Samples.

Blood collected from the tail vein, submandibular vein, or by cardiac puncture at the time of euthanization was centrifuged at 4° C. for 20 minutes at 16,000 RCF, after which serum fractions were collected and stored at −80° C. Saliva samples were collected with a micropipetter after stimulation of salivary flow by injecting each mouse intraperitoneally with 5.0 μg of carbachol (Sigma). Mice were placed into individual cages and fecal samples were collected and frozen at −80° C. until analysis. To prepare fecal material for Ab analysis, 100 mg of fecal pellets were dissolved in 400 μL PBS containing protease inhibitors. After extensive vortexing, samples were centrifuged at 16,000 RCF for 5 min at RT. Supernatant was collected for analysis. Samples of lung lavages were collected at the time of euthanization. Briefly, mice were anesthetized with isoflurane and exsanguinated by cardiac puncture. Pulmonary circulation was cleared by flushing 3 mL of PBS through the ventricles until the lungs visibly blanched. Blunt ended syringes were inserted into the trachea and tied off using sutures. Lungs of each animal were flushed with 1 mL of PBS. Bronchio-alveolar lavage (BAL) fluid was collected in microfuge tubes and frozen at −80° C.

Anti-RiVax™ and Anti-RTA Ab Analysis.

Levels of isotype and subclass anti-RiVax™ Ab in serum, saliva, lung lavage, and feces were measured by ELISA. Nunc Immulon 2HB polystyrene 96-well microtiter plates (ThermoFisher Scientific) coated with 100 μL RiVax™ (5 μg/mL) per well were incubated overnight at RT. To determine total IgA concentrations, plates were coated with 100 μL unlabeled goat anti-mouse IgA specific Ab (1 mg/mL) (Southern Biotechnology Assoc., Birmingham, Ala.). After blocking with PBS containing 0.15% Tween-20 and 1% bovine serum albumin (Amresco, Solon, Ohio), serial two-fold dilutions of serum or secretion samples were added in duplicate and plates were incubated overnight at RT. Plates were washed with PBS-Tween and incubated at RT for 4 h with the appropriate alkaline phosphatase-conjugated goat anti-mouse Ig isotype- or subclass-specific Ab (Southern Biotechnology). Plates were washed and developed with nitrophenyl phosphate substrate (Amresco) and the reaction was terminated by the addition of 100 μl/well of 2N NaOH. ELISA plates were read on a VersaMax microplate reader at 405 nm wavelength and analyzed with SoftMax Pro 5.4 (Molecular Devices, Sunnydale, Calif.). Concentrations of Ag-specific and total IgA Ab were calculated by interpolation of calibration curves generated using a mouse Ig reference serum (ICN Biomedicals, Aurora, Ill.). Salivary IgA responses are reported as the percentage of RiVax™-specific IgA in total IgA to compensate for variation in salivary flow rate.

Anti-RTA ELISAs were performed using previously established methods. Briefly, wells of Nunc Maxisorb F96 microtiter plates (ThermoFisher Scientific) were coated overnight with 100 μL RTA (1 μg/mL) in PBS (pH 7.4) prior to being treated with sera from the immunized mice. Horseradish peroxidase-labeled goat anti-mouse IgG-specific polyclonal Ab (Southern Biotechnology) was employed as a secondary detection reagent. ELISA plates were developed using the colorimetric detection substrate 3,3′,5,5′-tetramethylbenzidine (Kirkegaard & Perry Labs, Gaithersburg, Md.) and were analyzed using a SpectroMax 250 spectrophotometer and Softmax Pro 5.2 software (Molecular Devices).

Detection of Ricin-Neutralizing Ab.

Vero cell cytotoxicity assays were performed using a previously described method. Briefly, Vero cells were trypsinized, adjusted to approximately 5×104 cells per ml, and seeded (100 μl/well) into white bottom 96-well plates (Corning Life Sciences, Corning, N.Y.), and allowed to adhere overnight. Vero cells were treated with ricin (10 ng/ml), ricin:serum mixtures, or with culture medium (negative control) for 2 h at 37° C. Cells were washed to remove non-internalized ricin or ricin:serum mixtures, and were then incubated for 48 h. Cell viability was assessed using CellTiter-GLO reagent following manufacturer's protocol (Promega, Madison, Wis.). All treatments were performed in triplicate and 100% viability was defined as the average value obtained from wells in which cells were treated with culture medium only. Neutralizing data is presented as the reciprocal serum dilution required to protect 50% of ricin treated cells.

Ricin Challenge.

Groups of mice were immunized on days 0, 10, and 20 (and 34 for i.n.) by the i.d. or i.n. route with 0.5 μg of RiVax™ in the presence or absence of 1.0 μg of LT-IIb(T13I). On day 27 (i.d.) and 41 (i.n.), serum samples were analyzed for the presence of ricin-neutralizing Ab. One week later, mice were challenged with 10 LD50 (100 μg/kg) of ricin by intraperitoneal (i.p.) injection and monitored for 96 h for survival. As a surrogate marker of ricin intoxication, blood glucose measurements were taken just prior to ricin challenge and then every 24 h thereafter for 72 h. Blood samples were obtained from the tail vein and blood glucose levels were measured using an Aviva ACCU-CHEK handheld blood glucometer (Roche, Indianapolis, Ind.). Mice that became overtly moribund or when blood glucose levels fell below 25 mg/dl were euthanized. For statistical purposes, readings below the meter's limit of detection of 12 mg/dl were assigned that value.

Analysis of Skin Inflammation.

Mice immunized by the i.d. route with RiVax™ and adjuvants were observed every 24 h for reactivity at the immunization site. For gross morphologic analysis, indurations resulting from i d immunization were measured daily for 14 days using digital calipers and digital photographs were taken. Skin sections were collected for histological analysis from separate groups of mice at a time point 7 days after i.d. immunization. Skin sections were fixed overnight in 10% buffered formalin, embedded in paraffin, and cut into 5 μm sections. Unstained sections were employed for subsequent immunofluorescence analysis. Other sections were stained with hematoxylin and eosin (H&E) for light microscopic analysis. For light micrographs of H&E stained sections, images were taken using a Nikon Eclipse E600 Epifluorescence microscope at 40× and 200× magnification. Unstained sections were processed for immunostaining as previously described. Briefly, Ag retrieval of sections was performed by treating the sections for 20 min at 95° C. in 10 mM citrate buffer (pH 6). After cooling to RT, sections were washed in PBS and blocked with 2% milk for 1 h at RT, followed by additional washes in PBS. Sections were incubated overnight at 4° C. with dilutions (1:100) of primary rat anti-mouse CD45 and rabbit anti-mouse collagen 1 Ab in 1% milk. After washing in PBS, sections were incubated for 1 h at RT with 1:500 dilutions of chicken anti-rat Alexa647 and chicken anti-rabbit Alexa488 secondary Ab (Invitrogen). After washing in PBS, sections were mounted using SlowFade Gold mounting medium containing DAPI nuclear stain (Invitrogen) and analyzed using a Zeiss Axioimager fluorescence microscope at 200× magnification. Fifteen random images were obtained throughout each section and the number of CD45+ immune cells and the total number of nuclei were counted using Axiovision and ImageJ software.

Statistical Analysis.

Data were statistically analyzed using Excel 2008 (Microsoft, Redmond, Calif.) and Prism 5 (GraphPad Software, Inc., San Diego, Calif.). Unpaired Student's t-tests were performed to analyze differences between two groups and survival curves were analyzed using the Logrank test.

### Results

Intradermal Co-Administration of RTA Antigen with Either LT-IIb or LT-IIb(T13I)

I.d. Co-Administration of LT-IIb(T13I) with RiVax™ Enhances RiVax™-Specific Ab.

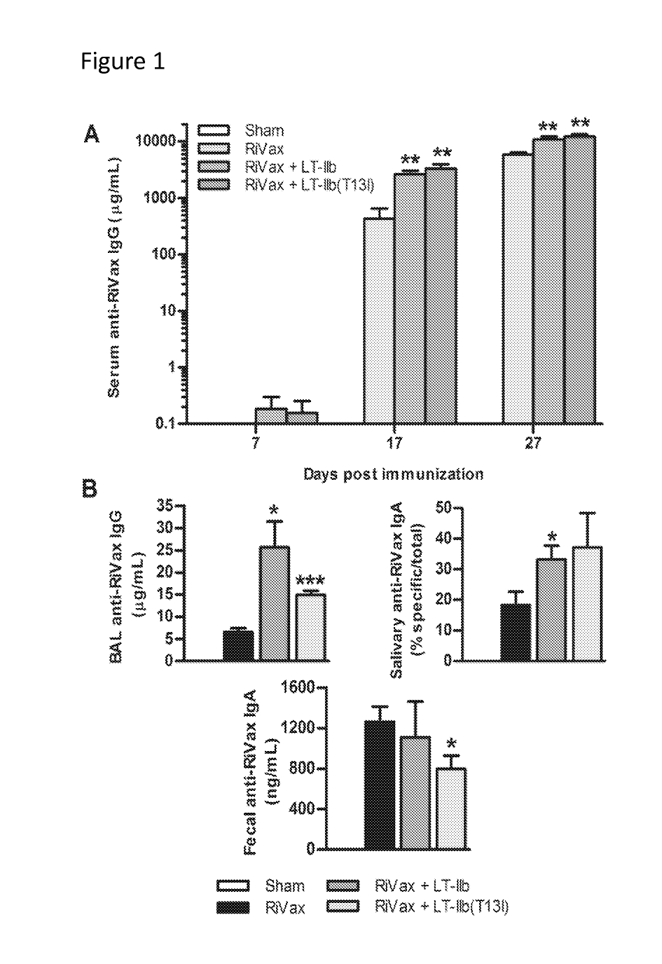

To evaluate the capacity of LT-IIb and detoxified LT-IIb(T13I) to enhance Ag-specific immune responses when administered by an intradermal (i.d.) route, mice were immunized with RiVax™ in the presence or absence of either adjuvant. We found that RiVax™ was moderately immunogenic when administered by the i.d. route. Measurable levels of anti-RiVax™ serum IgG were detected on days 17 and 27. In contrast, mice immunized with RiVax™ in the presence of LT-IIb or LT-IIb(T13I) produced on day 17 a seven to eight-fold increase in anti-RiVax™ IgG Ab and nearly a two-fold increase on day 27 in comparison to mice administered RiVax™ in the absence of adjuvant (FIG. 1A). Additionally, mice immunized with RiVax™ in combination with either LT-IIb or LT-IIb(T13I) had higher levels of anti-RiVax™ IgG and IgA in BAL fluid and saliva, in comparison to mice administered RaVax in the absence of adjuvant (FIG. 1B). While the average increase in anti-RiVax™ IgA induced by LT-IIb(T13I) was similar to the increase of anti-RiVax™ IgA induced by use of LT-IIb, the amount of variation in the LT-IIb(T13I) group precluded the attainment of significant difference from the control. Finally, neither LT-IIb or LT-IIb(T13I), when administered i.d. with RiVax™, were effective at enhancing RiVax™-specific IgA levels in fecal extracts in comparison to mice that had received only RiVax™. In fact, when compared to control group, the levels of anti-RiVax™ IgA were slightly lower in the LT-IIb(T13I) administered mice (FIG. 1B).

These data firmly demonstrated that LT-IIb and LT-IIb(T13I) are potent adjuvants for ricin antigen when administered by the i.d. route. In addition, the significant increase in salivary anti-RiVax™ IgA suggested that LT-IIb promotes Ag-specific mucosal immune responses in mice when administered in the skin.

I.d. Co-Administration of LT-IIb(T13I) with RiVax™ Enhances the Production of Ricin-Neutralizing Ab.

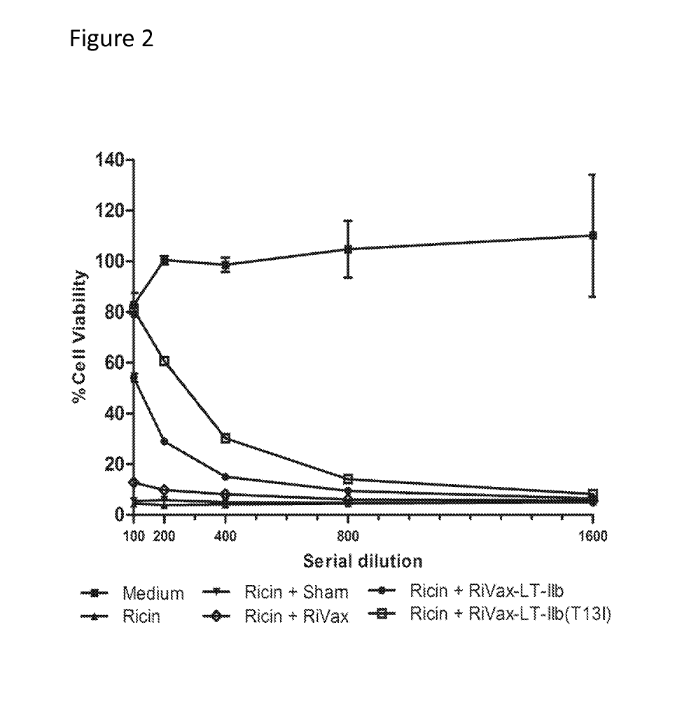

The production of ricin-neutralizing Ab is a hallmark of protection against ricin. To determine if the use of LT-IIb or LT-IIb(T13I) as adjuvants enhanced the production of ricin-neutralizing Ab in RiVax™-immunized mice, a well-established Vero cell in vitro cytotoxicity assay was employed (FIG. 2 and Table 1). Sera from mice immunized only with RiVax™ had no detectable toxin-neutralizing activity (TNA), despite elevated levels of RTA-specific Ab. This observation was not completely unexpected since neutralizing Ab constitute only a small fraction of the total Ag-specific Ab elicited by immunization with RiVax™. In contrast, ricin-neutralizing antibodies were detected in the sera of 80% of mice that had been immunized with RiVax™ in combination with LT-IIb or with LT-IIb(T13I). It is interesting to note that animals immunized solely with RiVax™ had no detectable TNA despite exhibiting RTA-specific titers that were comparable to those in mice that had been immunized with RiVax™-LT-IIb or RiVax™-LT-IIb(T13I). These data demonstrated that co-administration of LT-IIb or LT-IIb(T13I) with RiVax™ qualitatively and quantitatively enhances ricin-specific immune responses.

LT-IIb(T13I) Enhances Protective Immunity to Ricin when Co-Administered with RiVax™ in the Skin.

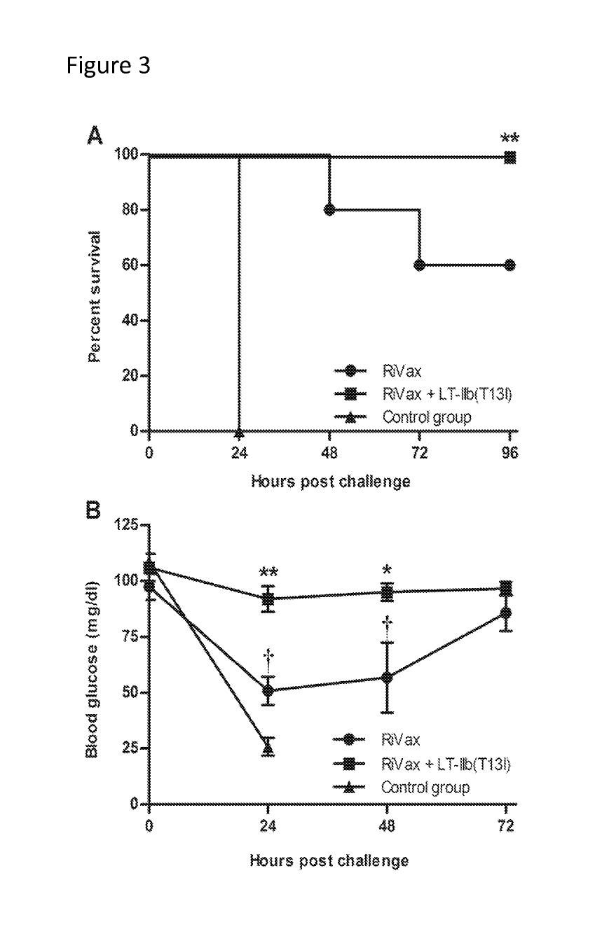

Next, the capacity of LT-IIb(T13I) to enhance the ability of RiVax™ to elicit protection against ricin toxin using an established mouse challenge model was investigated. Mice were immunized with RiVax™ (0.5 μg) or RiVax™ in combination with LT-IIb(T13I) (1.0 μg). Mice were boosted twice on days 10 and 20 using the same combination of Ag and adjuvant. As observed previously, only mice that received RiVax™ in combination with LT-IIb(T13I) produced detectable levels of ricin-neutralizing Ab (Table 2). To assess protective immunity, mice were challenged with 10 LD50 of ricin two weeks after the final immunization. Within 24 h after ricin challenge, all of the sham-immunized mice succumbed to intoxication. By 72 h, 40% of mice that been immunized solely with RiVax™ had died of ricin intoxication (FIG. 3A). In contrast, all of the RiVax™-LT-IIb(T13I) immunized mice survived the toxin challenge. As a quantitative measure of ricin intoxication and as a means to determine the effects of the toxin on basic metabolism, the onset of hypoglycemia in ricin-challenged mice was measured. RiVax™-LT-IIb(T13I) immunized mice experienced no statistical reduction in blood glucose levels following toxin exposure. In contrast, all RiVax™-immunized mice that survived the challenge experienced significant drops in blood glucose at time points of 72 h after challenge (FIG. 3B). These data established that LT-IIb(T13I) has the capacity to significantly enhance the protective ability of RiVax™.

LT-IIb(T13I) is Superior to an Aluminum Adjuvant when Administered Intradermally.

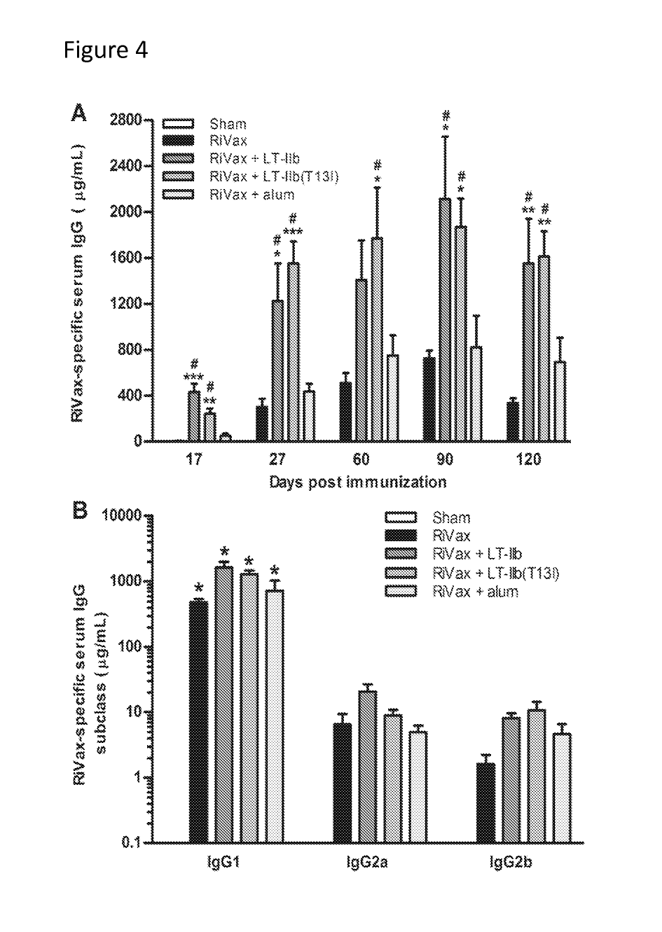

To compare the relative strength of the different adjuvants, mice were immunized with RiVax™ (0.5 μg) in the presence of LT-IIb, LT-IIb(T13I), or Imject® Alum, a commercially available adjuvant consisting of aluminum hydroxide and magnesium hydroxide. As expected, mice immunized with RiVax™ in combination with any of the adjuvants had higher levels of RiVax™-specific serum IgG Ab on day 17 than did mice immunized with RiVax™ in the absence of adjuvant. Mice which received RiVax™ in combination with LT-IIb or LT-IIb(T13I) produced significantly higher levels of anti-RiVax™ IgG Ab on days 17, 27, and 120, than did mice that received RiVax™ in combination with Imject® (FIG. 4A). On days 60 and 90, mice that received RiVax™ with LT-IIb or LT-IIb(T13I) produced significantly enhanced levels of serum anti-RiVax™ IgG in comparison to mice that had received only RiVax™, but not when compared to mice receiving RiVax™ in combination with Imject®. Overall, RiVax™ at this lower dose (0.5 μg) was poorly immunogenic. Mice that received RiVax™ in the presence of LT-IIb or LT-IIb(T13I), however, had significantly increased RiVax™-specific Ab production. By day 90, RiVax™-specific Ab levels continued to rise in all of the immunized groups. By day 120, however, the levels of RiVax™-specific Ab began to decline in all groups. Nonetheless, the RiVax™-specific Ab in mice receiving LT-IIb or LT-IIb(T13I) as adjuvants were significantly higher than those levels in mice receiving Imject® Alum, suggesting that LT-IIb and LT-IIb(T13I) induce long term Ag-specific immune responses.

Sera from immunized mice were also analyzed for the distribution of Ag-specific IgG subclasses. A significant enhancement of RiVax™-specific IgG1 and IgG2b Ab was observed in mice immunized with RiVax™ in combination with LT-IIb or LT-IIb(T13I) in comparison to the distribution of IgG1 and IgG2b in mice immunized solely with RiVax™ (FIG. 4B).

Collectively, these data indicated that, using our accelerated immunization model, LT-IIb and LT-IIb(T13I) were superior to the aluminum adjuvant at stimulating production of RiVax™-specific Ab when co-administered by the i.d. route. These experiments also established that the humoral immune response generated by RiVax™ is predominantly of the IgG1 subclass.

Local Inflammation is Reduced in Mice Immunized with LT-IIb(T13I).

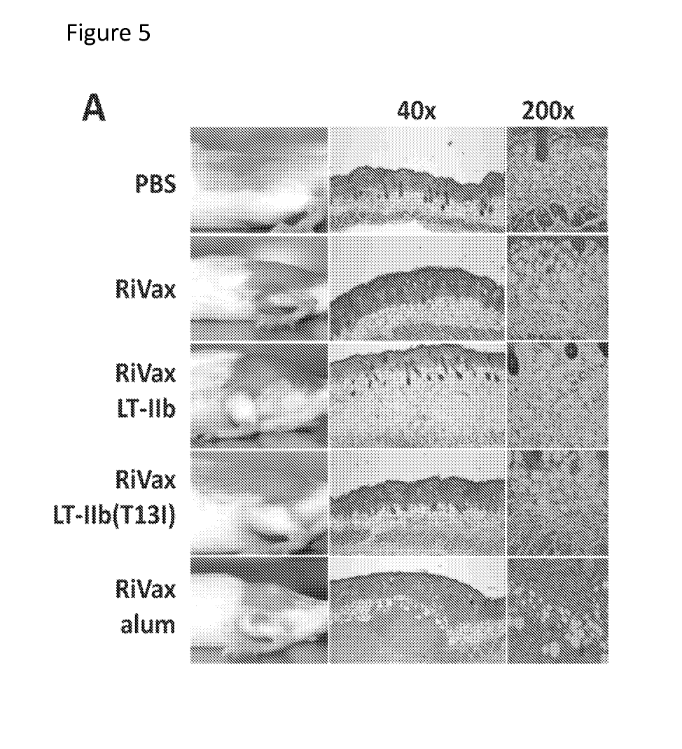

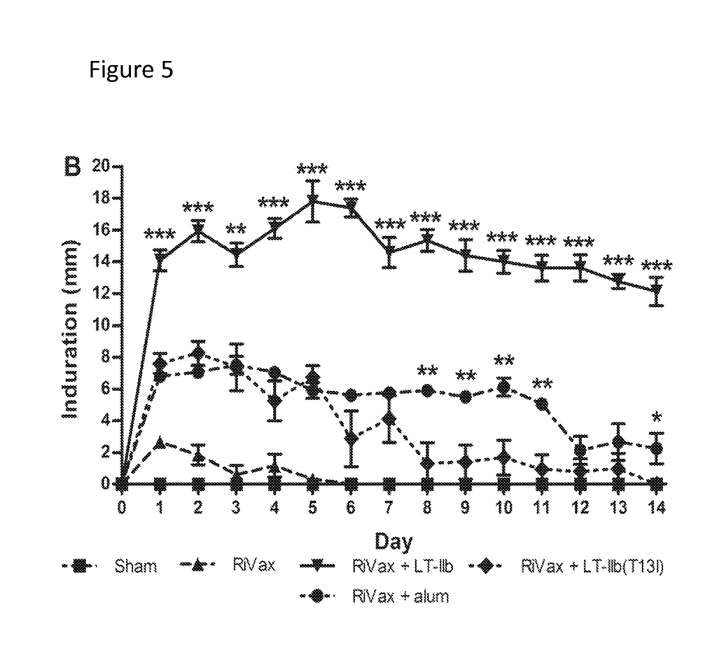

When adjuvants are employed, excessive and undesirable inflammation is often observed at the site of immunization. To assess the degree to which LT-IIb or LT-IIb(T13I) might induce inflammation at the site of injection, inflammatory responses at injection sites were monitored in i.d. immunized mice over the course of two weeks. RiVax™ induced a very low-inflammatory response evidenced by minor indurations observed in the skin that dissipated after a few days (FIG. 5). At 7 days post-immunization, mice administered RiVax™ in combination with LT-IIb had large indurations at sites of injection that were accompanied by extensive fluid accumulation and cellular infiltration, especially in the adipose layer of the skin (FIGS. 5A and B). In contrast, only minimal inflammatory responses were associated with injection of LT-IIb(T13I). Microscopic analysis of skin sections at the site of immunization confirmed that LT-IIb(T13I) was much less inflammatory than LT-IIb. Indeed, the inflammatory response induced by injection of LT-IIb(T13I) in combination with RiVax™ was comparable to the swelling produced by injection with RiVax™ in the absence of adjuvant (FIG. 5A). Substantial amounts of cellular infiltration with minor edema were observed in mice that received RiVax™ in combination with Imject® Alum.

LT-IIb and LT-IIb(T13I) also exhibited differences in the duration of the inflammatory responses. Whereas LT-IIb-associated inflammation persisted beyond 14 days, LT-IIb(T13I)-associated inflammation had fully subsided by day 8. In fact, during the first 7 days post-immunization, the amount of inflammation elicited by LT-IIb(T13I) was similar to that elicited by Imject® Alum. By the second week, however, LT-IIb(T13I)-associated inflammation was greatly reduced, whereas the indurations induced by Imject® Alum persisted (FIG. 5B).

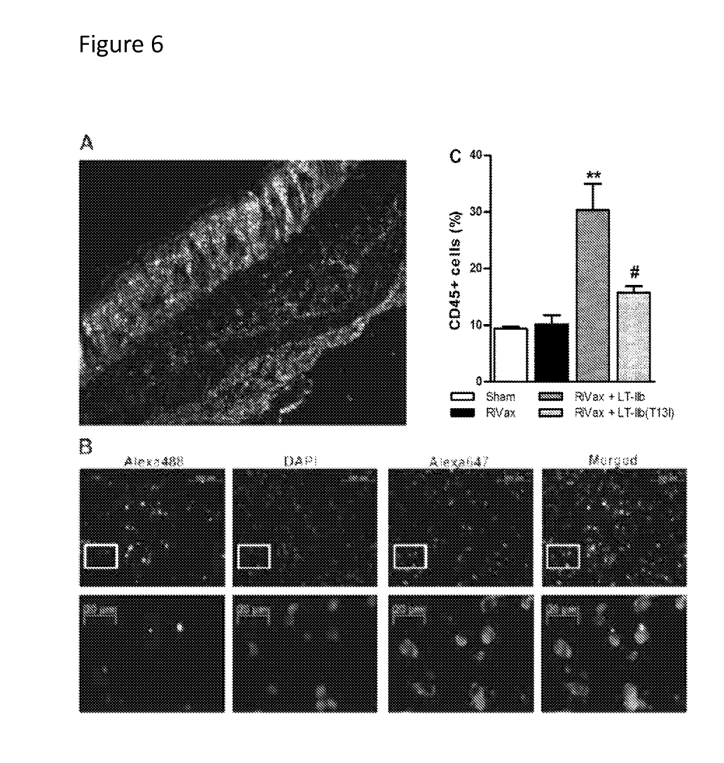

Inflammatory responses can be semi-quantified by enumerating the types and relative quantity of immune cells at the site of immunization. As a further means to quantify inflammation associated with LT-IIb or LT-IIb(T13I) administration, skin samples at the sites of immunization were analyzed by immunofluorescence staining (FIG. 6A). An enhanced number of CD45+ immune cells were observed at the injection sites of RiVax™-LT-IIb and RiVax™-LT-IIb(T13I) (30% and 16%, respectively) in comparison to the numbers of cells found in the sites that had received RiVax™ in the absence of adjuvant (FIGS. 6B and C). Additionally, the accumulation of immune cells induced by LT-IIb(T13I) was significantly less than the accumulation induced by LT-IIb. The excessive amount of cellular infiltration and autofluorescence induced by Imject® made it difficult to accurately quantify the numbers of immune cells in the skin of those mice. Thus, these data were omitted from the evaluation. Collectively, these data established that LT-IIb(T13I), a potent i.d. adjuvant, was only mildly inflammatory—unlike LT-I and LT-IIa, both of which induced significant amounts of inflammation at the administration site when delivered via the i.d. route.

Intranasal Co-Administration of RTA Antigen with Either LT-IIb or LT-IIb(T13I)

LT-IIb(T13I) Enhances the Immunogenicity and Protective Ability of RiVax™ when Co-Administered by the i.n. Route.

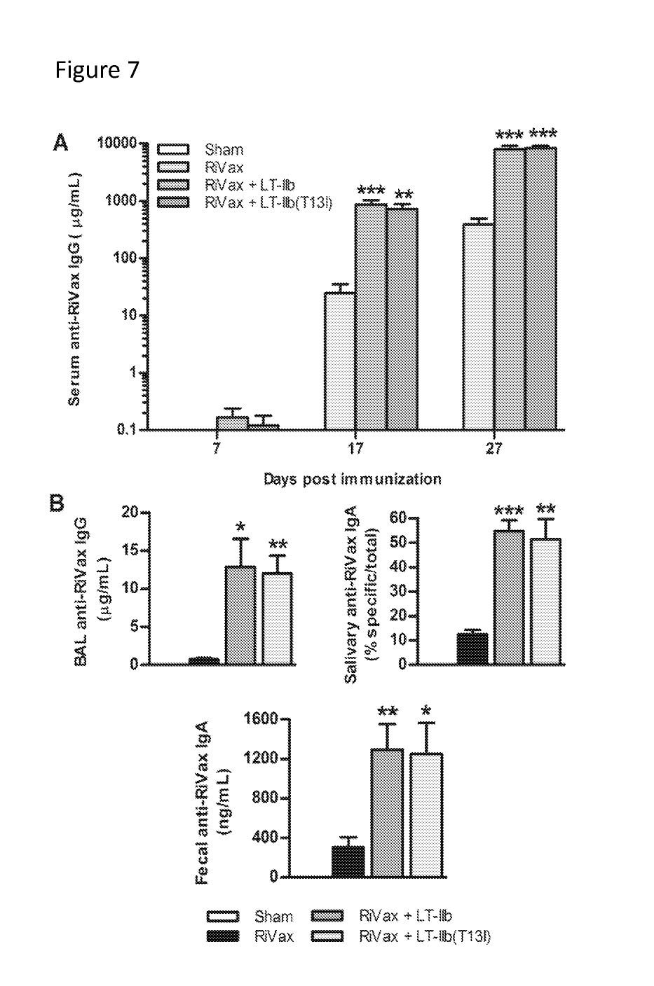

Due to endogenous regulatory mechanisms that suppress immune responses to Ag, induction of robust Ag-specific immune responses on mucosal surfaces often require the use of mucosal adjuvants. While RiVax™ has been shown to be immunogenic when administered by the i.d. and i.m. routes, the effectiveness of mucosal administration to enhance protective immune responses against ricin has not been sufficiently evaluated. To determine if LT-IIb or LT-IIb(T13I) augments immune responses against RiVax™ when administered by a mucosal route, mice were immunized intranasally with 5.0 μg of RiVax™ in the presence or absence of 1.0 μg LT-IIb or LT-IIb(T13I). I.n. immunization with RiVax™ elicited very low levels of Ag-specific Ab. In contrast, co-administration of RiVax™ with either LT-IIb or LT-IIb(T13I) significantly elevated RiVax™-specific Ab levels as early as 1 week after the booster immunization (day 17) (FIG. 7A). In addition, LT-IIb and LT-IIb(T13I) enhanced the anti-RiVax™ IgG and IgA Ab responses in BAL fluid, saliva, and fecal samples, each of which represent anatomical sites that are susceptible to ricin exposure (FIG. 7B). Overall, while RiVax™ is poorly immunogenic when delivered by the i.n. route, supplementation of RiVax™ with either LT-IIb or LT-IIb(T13I) induced mice to produce strong anti-RiVax™ Ab responses in the serum and at multiple mucosal sites.

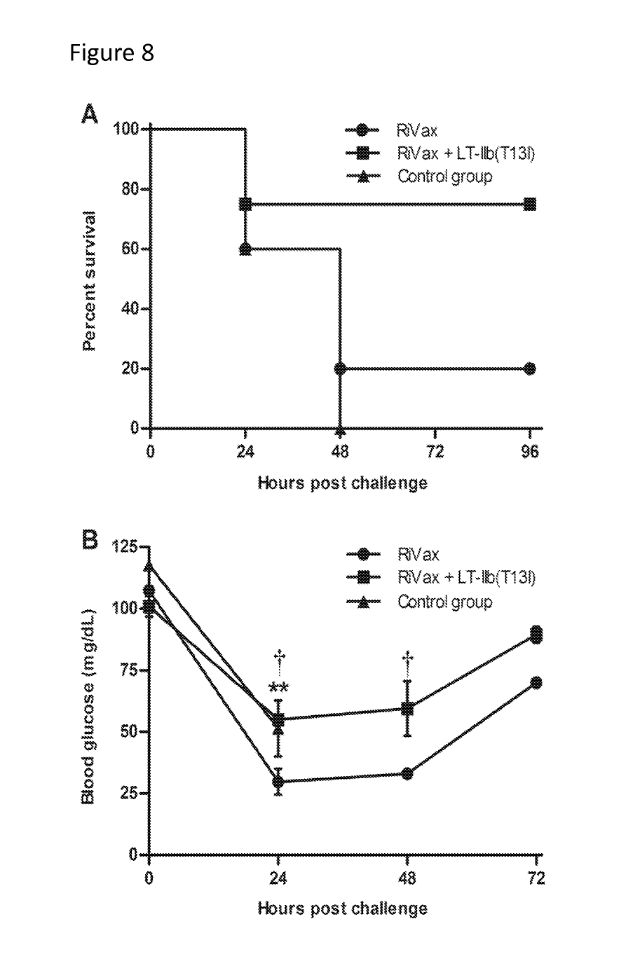

To explore the potential of LT-IIb(T13I) to provide a dose-sparing effect when administered intranasally, we repeated the i n immunization studies using a 10-fold lower dose of RiVax™ (0.5 μg). Mice that received the low dose vaccine by the i.n. route did not develop Ag-specific Ab as rapidly as mice that had been immunized by the i.d. route. An additional boost by the i.n. route was required to significantly raise anti-RTA titers. Two weeks after the final boost, immunized mice were challenged with 10 LD50 of ricin by i.p. injection and monitored for survival. All of the control mice and 80% of the mice that received 0.5 μg of RiVax™ by the i.n. route succumbed to intoxication by 48 h post challenge (FIG. 8A). Mice that received RiVax™ in combination with LT-IIb(T13I) had enhanced protection (75% survival). Although the mice that received RiVax™ with LT-IIb(T13I) exhibited greater protection against ricin challenge than the other cohorts in the study, blood glucose levels in these mice declined dramatically following ricin challenge, which demonstrated that the mice were only partially immune to the metabolic effects of the toxin (FIG. 8B). Taken together, these data indicated that combining RiVax™ with LT-IIb(T13I) enhanced protection when administered by the i.n. route, although the level of protection that is achieved is not as complete as in mice immunized with RiVax™ and LT-IIb(T13I) by the i.d. route.

In development of the present invention, we evaluated the capacity of LT-IIb(T13I), a detoxified LT-IIb HLT, to augment Ag-specific immune responses against RiVax™, a candidate ricin toxin vaccine Ag. When co-administered via the i.d. route with high (5.0 μg) and low (0.5 μg) doses of RiVax™, LT-IIb(T13I) significantly enhanced anti-RiVax™ serum IgG levels in comparison to the levels in mice that received RiVax™ alone. Additionally, LT-IIb(T13I) enhanced RiVax™-specific IgG Ab in the lungs, thus indicating the likelihood of enhanced protection at this important mucosal surface. Given the lethality of ricin, the capacity of LT-IIb(T13I) to rapidly elevate the levels of anti-RiVax™ Ab in the serum and in the lungs using a minimal immunization regimen provides a strong advantage over other adjuvants that have been evaluated. Importantly, in comparison to the use of RiVax™ alone, LT-IIb(T13I) also unexpectedly and preferentially stimulated the production of ricin-neutralizing Ab, which was correlated with a strong augmentation in protective immunity to ricin challenge.

Only a very small portion of the total RTA-specific antibodies elicited by RiVax™ immunization has neutralizing activity. These data indicate that methods to increase the overall amount of RTA-specific antibodies are less important for immune protection against ricin intoxication than are methods that increase the amount of RTA neutralizing antibodies. The observation that LT-IIb(T13I) skewed the production of antibodies in favor of ricin-neutralizing in comparison to the minor production of ricin-neutralizing Ab in mice immunized solely with RiVax™ has important implications for the immunomodulatory utility of that adjuvant. Indeed, the mechanism(s) by which LT-IIb(T13I) augments ricin-neutralizing Ab when employed as an adjuvant is unclear. LT-IIb(T13I) may preferentially promote the production of Ab to one or more neutralizing epitopes on RiVax™. Alternatively, LT-IIb(T13I), by virtue of its ability to bind to Ag-presenting cells, may accelerate Ag uptake, processing, or presentation. Or, LT-IIb(T13I) might accelerate B cell affinity maturation. Any one or combination of those mechanisms could augment the production of antibodies to neutralizing epitopes that would be ignored or minimally processed by immune cells in mice that did not receive the adjuvant.

The precise molecular mechanisms by which LT-IIb and LT-IIb(T13I) enhance production of cytokines and/or chemokines that favor production and affinity maturation of Ab have not been well-described. Interleukin-6 (IL-6), a potent B cell differentiation factor that is produced by many cell types, drives B cell maturation and stimulates Ab production. Indeed, LT-IIb induces robust production of IL-6 in several cell populations including mononuclear cells and lymphocytes. Whether LT-IIb(T13I)-induced cytokines/chemokines influence immune functions locally in the skin or within regional draining lymph nodes to enhance Ag-specific immune responses remains to be determined.

Co-administration of RiVax™ with detoxified LT-IIb(T13I) by either the i.d. route or the i.n. route enhanced protection to a lethal systemic ricin challenge, in comparison to the level of protection observed in mice that had received only RiVax™. A low dose of RiVax™ in combination with LT-IIb(T13I) imparted 100% survival against i.p. challenge with ricin. Importantly, the co-administration of LT-IIb(T13I) with RiVax™ provided complete protection against ricin intoxication when administered in the dermal layer of the skin. Mice that received LT-IIb(T13I) in combination with RiVax™ and that subsequently were challenged with ricin exhibited no significant alterations in blood glucose levels. In contrast, mice that were immunized only with RiVax™ had significantly lower level of protection. Prior to succumbing, these mice also exhibited a sharp drop in the levels of blood glucose, which indicated a severe effect of ricin challenge on normal metabolism.

To optimize the willingness of the recipient to receive an initial vaccination and potential boosters, a vaccine must be tolerated by the recipient. Although alum has a long history of success as an i.m. adjuvant, this adjuvant can often induce long-lasting granulomas at the injection site and elicit local allergic reactions. In comparison to alum, LT-IIb(T13I) elaborated a decreased propensity to promote inflammation at the site of immunization. Large depots in the dermis and subcutaneous regions that contained very large numbers of cells were observed in the skin of mice that received RiVax™ adsorbed to alum by the i.d. route. In contrast, less edema and cellular infiltrates were observed at the sites of i.d. immunization of mice receiving RiVax™ with LT-IIb(T13I). In fact, the skin sections from these mice were largely indistinguishable from skin sections obtained from mice that had received only RiVax™. While the mechanisms by which LT-IIb(T13I) augments Ag-specific immune responses in the skin have not been fully elucidated, it is feasible that LT-IIb(T13I), by its inability to increase cAMP in cells (unlike wt LT-IIb), fails to induce inflammatory cytokines at the site of immunization. Another possibility is that LT-IIb(T13I) induces infiltration of one or more types of immune cells into the site that down-regulate potential inflammatory responses.

Although immunization by either route induced similar levels of Ag-specific IgG Ab, it is surprising that immunization with RiVax™ and LT-IIb(T13I) by the i.n. route was less effective than the i.d. route at stimulating protection against ricin intoxication.

This disclosure demonstrates that LT-IIb and the detoxified mutant LT-IIb(T13I) are potent i.d. adjuvants when co-administered with RiVax™, a vaccine candidate against ricin. LT-IIb and LT-IIb(T13I) not only enhanced the production of anti-RiVax™ Ab when administered by the i.d. route, but also increased the levels of ricin-neutralizing Ab in the serum. When administered in the skin, LT-IIb(T13I) was much less inflammatory than either LT-IIb or alum. Importantly, LT-IIb(T13I) elevated the ability of RiVax™ to induce protective immunity to a lethal challenge of ricin. Taken together, these data support the potential use of LT-IIb(T13I) as an effective and safe next-generation i.d. adjuvant.

TABLE 1
Detection of ricin-neutralizing antibody titers after i.d. immunization
with RiVax ™ and LT-IIb enterotoxins

| Group and Mouse # | RTA Titers^{a} | Neutralizing Titers^{b} |
|---|---|---|
| Sham | 0 | 0 |
| RiVax ™ #1 | 64000 | 0 |
| RiVax ™ #2 | 64000 | 0 |
| RiVax ™ #3 | 32000 | 0 |
| RiVax ™ #4 | 128000 | 0 |
| RiVax ™ #5 | 64000 | 0 |
| RiVax ™ + LT-IIb #1 | 128000 | 50 |
| RiVax ™ + LT-IIb #2 | 64000 | 0 |
| RiVax ™ + LT-IIb #3 | 64000 | 100 |
| RiVax ™ + LT-IIb #4 | 64000 | 50 |
| RiVax ™ + LT-IIb #5 | 128000 | 100 |
| RiVax ™ + LT-IIb(T13I) #1 | 128000 | 50 |
| RiVax ™ + LT-IIb(T13I) #2 | 128000 | 800 |
| RiVax ™ + LT-IIb(T13I) #3 | 64000 | 50 |
| RiVax ™ + LT-IIb(T13I) #4 | 128000 | 200 |
| RiVax ™ + LT-IIb(T13I) #5 | 64000 | 0 |

^{a}Values are reciprocal endpoint titers, as described in Materials and Methods;
^{b}Neutralizing endpoint titers were determined in a Vero cell cytotoxicity assay, as described in Materials and Methods.

TABLE 2
Pre-challenge determination of ricin-neutralizing antibodies after low
dose i.d. immunizations with RiVax ™ and LT-IIb(T13I) adjuvant.

| | | Neutralizing |
|---|---|---|
| Group and Mouse # | RTA Titers^{a} | Titers^{b} |
| RiVax ™ #1 | 0 | 0 |
| RiVax ™ #2 | 1000 | 0 |
| RiVax ™ #3 | 8000 | 0 |
| RiVax ™ #4 | 0 | 0 |
| RiVax ™ #5 | 0 | 0 |
| RiVax ™ + LT-IIb(T13I) | 16000 | 0 |
| #1 | | |
| RiVax ™ + LT-IIb(T13I) | 16000 | 25 |
| #2 | | |
| RiVax ™ + LT-IIb(T13I) | 16000 | 0 |
| #3 | | |
| RiVax ™ + LT-IIb(T13I) | 8000 | 50 |
| #4 | | |
| RiVax ™ + LT-IIb(T13I) | 32000 | 25 |
| #5 | | |
| Control #1 | 0 | 0 |
| Control #2 | 0 | 0 |
| Control #3 | 0 | 0 |
| Control #4 | 0 | 0 |
| Control #5 | 0 | 0 |

^{a}Values are reciprocal endpoint titers, as described in Materials and Methods;
^{b}Neutralizing endpoint titers were determined in a Vero cell cytotoxicity assay, as described in Materials and Methods.

SEQ ID NO 1
LENGTH: 122
TYPE: PRT
ORGANISM: Escherichia coli
OTHER INFORMATION: LT-IIb B polypeptide
SEQUENCE: 1
Met Ser Phe Lys Lys Ile Ile Lys Ala Phe Val Ile
Met Ala Ala Leu Val Ser Val Gln Ala His Ala Gly
Ala Ser Gln Phe Phe Lys Asp Asn Cys Asn Arg Thr
Thr Ala Ser Leu Val Glu Gly Val Glu Leu Thr Lys
Tyr Ile Ser Asp Ile Asn Asn Asn Thr Asp Gly Met
Tyr Val Val Ser Ser Thr Gly Gly Val Trp Arg Ile
Ser Arg Ala Lys Asp Tyr Pro Asp Asn Val Met Thr
Ala Glu Met Arg Lys Ile Ala Met Ala Ala Val Leu
Ser Gly Met Arg Val Asn Met Cys Ala Ser Pro Ala
Ser Ser Pro Asn Val Ile Trp Ala Ile Glu Leu Glu
Ala Glu
SEQ ID NO 2
LENGTH: 99
TYPE: PRT
ORGANISM: Escherichia coli
OTHER INFORMATION: LT-IIb B mature polypeptide
SEQUENCE: 2
Gly Ala Ser Gln Phe Phe Lys Asp Asn Cys Asn Arg
Thr Thr Ala Ser Leu Val Glu Gly Val Glu Leu Thr
Lys Tyr Ile Ser Asp Ile Asn Asn Asn Thr Asp Gly
Met Tyr Val Val Ser Ser Thr Gly Gly Val Trp Arg
Ile Ser Arg Ala Lys Asp Tyr Pro Asp Asn Val Met
Thr Ala Glu Met Arg Lys Ile Ala Met Ala Ala Val
Leu Ser Gly Met Arg Val Asn Met Cys Ala Ser Pro
Ala Ser Ser Pro Asn Val Ile Trp Ala Ile Glu Leu
Glu Ala Glu
SEQ ID NO 3
LENGTH: 99
TYPE: PRT
ORGANISM: Escherichia coli
OTHER INFORMATION
SEQUENCE: 3
Gly Ala Ser Gln Phe Phe Lys Asp Asn Cys Asn Arg
Ile Thr Ala Ser Leu Val Glu Gly Val Glu Leu Thr
Lys Tyr Ile Ser Asp Ile Asn Asn Asn Thr Asp Gly
Met Tyr Val Val Ser Ser Thr Gly Gly Val Trp Arg
Ile Ser Arg Ala Lys Asp Tyr Pro Asp Asn Val Met
Thr Ala Glu Met Arg Lys Ile Ala Met Ala Ala Val
Leu Ser Gly Met Arg Val Asn Met Cys Ala Ser Pro
Ala Ser Ser Pro Asn Val Ile Trp Ala Ile Glu Leu
Glu Ala Glu
Note:
the first 23 amino acids of SEQ ID NO: 1 represent the leader sequence.

1. A method for enhancing an immune response to an antigen in an individual comprising administering intradermally to the individual a composition comprising an effective amount of:
(a) LT-IIb(T13I) enterotoxin, and
(b) a toxin,
wherein the resulting immune response is enriched for toxin neutralizing antibodies.

2. The method of claim 1, wherein the toxin is a ricin antigen.

3. The method of claim 1, wherein the ricin toxin is the A subunit of ricin or a non-toxic variant of the A subunit.

4. The method of claim 1, wherein the enterotoxin and the toxin are administered simultaneously.

5. The method of claim 1, wherein the enterotoxin and the toxin are administered sequentially.
